(19) United States
(12) Patent Application Publication
(10) Pub. No.: US 2025/0258172 A1
(43) Pub. Date: Aug. 14, 2025

(54) METHOD OF CHARACTERIZING A RECOMBINANT VIRUS

(71) Applicant: Arizona Board of Regents on Behalf of the University of Arizona, Tucson, AZ (US)

(72) Inventors: Michael T. Marty, Tucson, AZ (US); Marius M. Kostelic, Tucson, AZ (US)

(21) Appl. No.: 18/866,934

(22) PCT Filed: May 22, 2023
(86) PCT No.: PCT/US2023/067309

§ 371 Date: Nov. 18, 2024

Related U.S. Application Data
(60) Provisional application No. 63/344,524, filed on May 20, 2022.

(51) Int. Cl. G01N 33/569; G01N 33/68; H01J 49/16

(52) U.S. Cl. CPC G01N 33/56983; G01N 33/6851; H01J 49/167; G01N 2333/015

(57) ABSTRACT

A first method of analyzing recombinant viruses is provided. The method comprises infusing a sample of recombinant viruses into a charge-detection mass spectrometer, the spectrometer comprising an electrospray needle that is maintained at a first temperature for a first period of time; detecting a first analyte during the first period of time; maintaining the electrospray needle at a second temperature higher than the first temperature for a second period of time; and detecting the first analyte during the second period of time. A second method of using the first method to compare stability of two or more distinct viral suspensions is also provided.

### CROSS REFERENCE TO RELATED APPLICATIONS

This application claims priority to U.S. Application No. 63/344,524, filed on May 20, 2022, the contents of which are hereby incorporated by reference in its entirety.

### STATEMENT OF FEDERALLY SPONSORED RESEARCH AND DEVELOPMENT

This invention was made with government support under Grant No. CHE-184530 awarded by the National Science Foundation. The government has certain rights in the invention.

### FIELD OF THE INVENTION

The field of the invention relates generally to compositions comprising Adeno-associated viruses (AAVs) and methods for characterizing the stability of the AAVs.

### BACKGROUND

Adeno-associated viral (AAV) capsids have emerged as gene therapy and vaccine delivery systems. Differential scanning fluorimetry or differential scanning calorimetry are commonly used to measure the thermal stability of AAVs, but these global methods are unable to distinguish the stability of different AAV subpopulations in the same sample.

### DESCRIPTION

### Definitions

For the purposes of promoting an understanding of the principles of the invention, reference will now be made to certain embodiments and specific language will be used to describe the same. It will nevertheless be understood that no limitation of the scope of the invention is thereby intended, and alterations and modifications in the illustrated invention, and further applications of the principles of the invention as illustrated therein are herein contemplated as would normally occur to one skilled in the art to which the invention relates.

Unless defined otherwise, all technical and scientific terms used herein have the same meaning as commonly understood by one of ordinary skill in the art to which this invention pertains.

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used).

The use of “or” means “and/or” unless stated otherwise.

The use of “a” or “an” herein means “one or more” unless stated otherwise or where the use of “one or more” is clearly inappropriate.

The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

As used herein, the term “about” refers to a ±10% variation from the nominal value. It is to be understood that such a variation is always included in any given value provided herein, whether or not it is specifically referred to.

As used herein, a “recombinant viral particle” refers to a virus that is generated to incorporate modification(s) introduced (e.g., by using recombinant DNA technology) into a gene/locus (or multiple loci). Such modifications include, but are not limited to, incorporation of genes or portions thereof that can be expressed in cells that are infected with the recombinant viral particle.

As used herein, “empty capsid” refers to a viral particle that lacks a nucleic acid cargo. AAV vectors are known to generate quantities (sometimes, substantial quantities) of empty capsids when propagated in cell culture.

As used herein, “embedded cargo” refers to material (e.g., a polynucleotide, a protein, a salt, a small molecule) that is housed within a viral capsid.

As used herein, an “ionized component” refers to viral particles or a portion thereof that has a net positive or net negative charge.

As used herein, a “fully-filled virion” refers to a viral particle that comprises a capsid that houses a polynucleotide, the polynucleotide of a size that is within a range considered “normal” for the virus from which the capsid was derived or from the intended cargo (e.g., a recombinant polynucleotide). The polynucleotide comprises an AAV genome encoding proteins for replicating AAV viruses or it may comprise exogenous (e.g., man-made) nucleic acid.

As used herein, a “partially-filled virion” refers to a viral particle that comprises a capsid that houses a polynucleotide, the polynucleotide of a size that is smaller than the lower limit considered “normal” for the virus from which the capsid was derived.

As used herein, an “over-filled virion” refers to a viral particle that comprises a capsid that houses a polynucleotide, the polynucleotide of a size that is larger than the upper limit considered “normal” for the virus from which the capsid was derived or having more than one copy of the intended polynucleotide.

As used herein, a “virion capsid protein” refers to a protein that is a component of a fully assembled viral capsid.

As used herein, an “environmental stress agent” refers to a reagent (e.g., an acid, a base, an oxidizing reagent, a surfactant) or a treatment (e.g., storage time, temperature (e.g., a nonphysiological temperature), agitation) that can cause instability of viral particles or components thereof. Without being bound by theory, it is believed environmental stress agents may influence the tertiary structure of a capsid protein and/or a polynucleotide housed within a capsid.

As used herein, an “Adeno-associated viral particle” refers to a protein shell surrounding and protecting a small, single-stranded DNA genome of about 1 to about 10 kilobases (kb), or about 2 to about 8 kb, or about 3 to about 6 kb, or about 4 to about 5 kb, or about 4.5 to about 4.9 kb, or about 4.7 kb, or about 4.8 kb. AAV belongs to the parvovirus family and is dependent on co-infection with other viruses, mainly adenoviruses, in order to replicate.

As used herein, a “clone” refers to the aggregate of genetically identical organisms (e.g., viruses) asexually produced by or from a single progenitor cell or organism.

As used herein, a “nucleic acid cargo” refers to a polynucleotide fully or partially encased by viral capsid protein.

Adeno-associated viruses are one of the promising potential delivery systems for gene therapeutics and vaccines. Originally discovered as a contaminant of adenovirus preparations, they produce a protein shell that surrounds a small (e.g., about 4.8 kilobase) single-stranded DNA genome. The protein shell functions as a delivery vehicle to bind target cells (e.g., human cells), where they bind and are internalized in the cells. Once inside, the viral genes are expressed to cause replication of the viral DNA, production of the capsid proteins that make up the protein shell, and assembly of the mature viruses.

Recombinant AAV (rAAV) lack the viral DNA and, thus, are typically produced by co-infecting cells with rAAV and “helper” AAV. The helper AAV provide the viral genome needed to produce the viral proteins (e.g., the replicase and the capsid proteins) and assemble the rAAV. A number of strains, species and serotypes of AAV have been discovered, each having its own unique properties. Commonly found in rAAV preparations are several types of viral particles-partially filled virions, fully filled virions, and over filled virions, each of which can be individually identified and optionally quantified in the methods of the present disclosure.

In one aspect, the present disclosure provides a first method for characterizing AAVs. The inventive first method combines charge-detection mass spectrometry (“CD-MS”) with variable-temperature electrospray analysis to provide better discrimination between the individual stabilities of multiple forms of the viral particles that may be present in an AAV preparation. This increased discrimination provides the operator a unique ability to determine the stability of the fully assembled viral particles even if the AAV preparation comprises partially filled virions, over filled virions, and/or various assemblies of capsid proteins lacking a nucleic acid cargo.

The methods disclosed herein can be used to characterize and optionally select viral clones for a variety of useful biological or medical applications including, for example, gene therapy, vaccine development, creating model systems (e.g., animals) for pharmaceutical or medical device research and development.

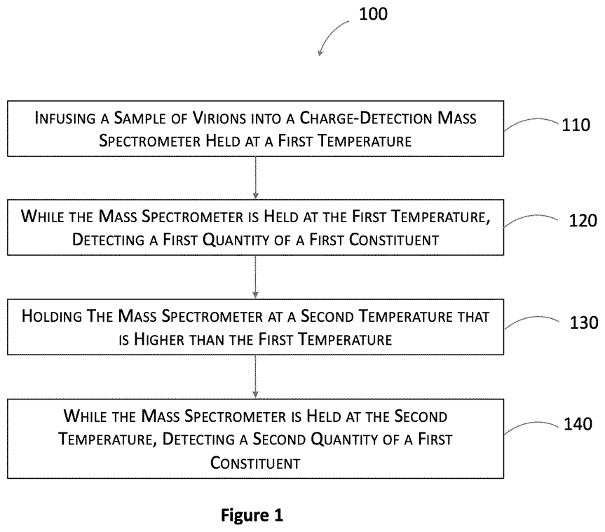

Turning to the drawings, FIG. 1 shows a block diagram of one embodiment of a first method 100 according to the present disclosure. The first method 100 comprises a step 110 of infusing a preparation of viruses (e.g., AAVs and/or constituents thereof) into a charge-detection mass spectrometer that is held at a first temperature (e.g., the electrospray needle of the mass spectrometer is held at the temperature). The mass spectrometer is held at the first temperature for a period of time to permit detection of ionic species of viral constituents (e.g., fully filled virions, partially filled virions, over-filled virions, and/or partially assembled capsids that comprise capsid proteins but lack a nucleic acid cargo) that are present in the sample in the mass spectrometer. Contemporary CD-MS instruments may include the capability to control the temperature of the electrospray needle as the sample is passed therethrough, as discussed in the Examples.

In some embodiments, the first method 100 further comprises the step 120 of detecting a first analyte (e.g., a viral constituent such a fully filled virion, a partially filled virion, an over filled virion, and/or a partially assembled capsid that comprises capsid proteins but lacks a nucleic acid cargo) as a while the sample in the mass spectrometer is held at the first temperature. Generally, the viral constituents of the sample are detected and identified by their respective mass (m), charge (z) and/or their respective charge/mass (m/z) ratio. Fully filled viral rAAV constituents in the sample have a molecular mass that is similar to the AAV used to produce them. In addition, partially filled and over filled viral constituents are also identified by their molecular masses, which are smaller than fully filled constituents and larger than fully filled constituents, respectively.

In some embodiments, the first method further comprises the step 130 of holding the mass spectrometer at a second temperature that is higher than the first temperature. Holding the mass spectrometer at a second temperature comprises holding the mass spectrometer at a second temperature for a second period of time. In any embodiment, the second period of time can be shorter than the first period of time, longer than the first period of time, or equal to the first period of time.

In some embodiments, the first method further comprises the step 140 of detecting the first viral constituent while the sample in the mass spectrometer is held at the second temperature.

In any implementation, the first method can further comprise, after the first period of time and the second period of time, maintaining the electrospray needle at a third temperature for a third period of time while the portion is disposed in the electrospray needle; and, during the third period of time, detecting the first analyte. In these implementations, the third temperature is higher than the second temperature.

In any implementation of the first method, detecting the first analyte can further comprise quantifying the first analyte.

In any of the above implementations, the first method can further comprise; during the first period of time, the second period of time or the third period of time, detecting a second analyte having a second mass-to-charge (m/z) ratio and having a second charge (z). Optionally, this first method can further comprise quantifying the second analyte.

In any of the above implementations, the first method can further comprise; during the first period of time, the second period of time or the third period of time, detecting a third analyte having a third mass-to-charge (m/z) ratio and having a third charge (z). Optionally, this first method can further comprise quantifying the third analyte.

In any of the above implementations of the first method, the first analyte, second analyte, and/or the third analyte each can be an ionized analyte selected from a group consisting of a fully filled virion, a partially filled virion, an over filled virion and a partially assembled capsid that comprises capsid proteins but lacks a nucleic acid cargo.

In any implementation of the first method, the first temperature is greater than or equal to 0° C. In any implementation of the first method, the second or the third temperature can be less than or equal to 100° C. A person having ordinary skill in the art, in view of the present disclosure, will recognize the temperatures used in the first method will be selected at least 1) to permit detection of sufficient quantities of ionized analytes at the specified temperatures and 2) preferably, include temperatures that span a range of temperatures from lower temperatures, where all components are relatively stable, to higher temperatures, where some or all components are relatively unstable and may dissociate.

In any of the above implementations, the first method can further comprise, prior to the infusing the sample into the mass spectrometer, exposing the sample to (e.g., contacting the sample with) an effective amount of an environmental stress agent to destabilize at least a fraction of the recombinant the viral particles. An effective amount can be determined, for example, by measuring the viability (e.g., ability to infect a host cell) of the stressed virus preparation compared to a non-stressed preparation. An effective amount can also be determined by quantifying the fully filled virions in a sample exposed to an environmental stress agent compared to an identical sample not exposed to the environmental stress agent. In these implementations, the first method further comprises, while the portion of the sample is disposed in the electrospray needle, exposing the sample to the effective amount of the environmental stress agent. In these implementations, the environmental stress agent can be selected from a group consisting of an acid, a base, a salt, an oxidizer reagent, and a combination of any two or more of the foregoing environmental stress agents.

In any of the above implementations, first period of time is about 30 seconds to about 60 minutes. In any of the above implementations, first period of time may be about 1 minute to about 55 minutes. In any of the above implementations, first period of time may be about 2 minutes to about 50 minutes. In any of the above implementations, first period of time may be about 3 minutes to about 45 minutes. In any of the above implementations, first period of time may be about 4 minutes to about 40 minutes. In any of the above implementations, first period of time may be about 5 minutes to about 35 minutes. In any of the above implementations, first period of time may be about 6 minutes to about 30 minutes. In any of the above implementations, first period of time may be about 7 minutes to about 25 minutes. In any of the above implementations, first period of time may be about 8 minutes to about 20 minutes. In any of the above implementations, first period of time may be about 9 minutes to about 18 minutes. In any of the above implementations, first period of time may be about 10 minutes to about 15 minutes. In any of the above implementations, first period of time may be about 5 minutes. In any of the above implementations, first period of time may be about 10 minutes.

In any of the above implementations, second period of time is about 30 seconds to about 60 minutes. In any of the above implementations, second period of time may be about 1 minute to about 55 minutes. In any of the above implementations, second period of time may be about 2 minutes to about 50 minutes. In any of the above implementations, second period of time may be about 3 minutes to about 45 minutes. In any of the above implementations, second period of time may be about 4 minutes to about 40 minutes. In any of the above implementations, second period of time may be about 5 minutes to about 35 minutes. In any of the above implementations, second period of time may be about 6 minutes to about 30 minutes. In any of the above implementations, second period of time may be about 7 minutes to about 25 minutes. In any of the above implementations, second period of time may be about 8 minutes to about 20 minutes. In any of the above implementations, second period of time may be about 9 minutes to about 18 minutes. In any of the above implementations, second period of time may be about 10 minutes to about 15 minutes. In any of the above implementations, second period of time may be about 5 minutes. In any of the above implementations, second period of time may be about 10 minutes.

In any of the above implementations, third period of time is about 30 seconds to about 60 minutes. In any of the above implementations, third period of time may be about 1 minute to about 55 minutes. In any of the above implementations, third period of time may be about 2 minutes to about 50 minutes. In any of the above implementations, third period of time may be about 3 minutes to about 45 minutes. In any of the above implementations, third period of time may be about 4 minutes to about 40 minutes. In any of the above implementations, third period of time may be about 5 minutes to about 35 minutes. In any of the above implementations, third period of time may be about 6 minutes to about 30 minutes. In any of the above implementations, third period of time may be about 7 minutes to about 25 minutes. In any of the above implementations, third period of time may be about 8 minutes to about 20 minutes. In any of the above implementations, third period of time may be about 9 minutes to about 18 minutes. In any of the above implementations, third period of time may be about 10 minutes to about 15 minutes. In any of the above implementations, third period of time may be about 5 minutes. In any of the above implementations, third period of time may be about 10 minutes.

In any of the above implementations of the first method, the recombinant viral particles comprise Adeno-associated viral particles.

In another aspect, the present disclosure provides a second method of selecting a recombinant viral clone for gene therapy use. The second method comprises analyzing a first suspension of recombinant viral particles and a second suspension of viral particles using any implementation of the first method disclosed herein. The first and second viral suspensions are analyzed identically to detect at least a first analyte in each suspension as disclosed herein. Optionally, the first and second suspensions can further be analyzed to detect a second analyte (if present) and/or a third analyte (if present), and/or a fourth analyte (if present). The first analyte, second analyte, third analyte, and fourth analyte can be selected from a group consisting of a fully filled virion, a partially filled virion, an over filled virion and a partially assembled capsid that comprises capsid proteins but lacks a nucleic acid cargo.

In some aspects, in addition to the steps of the first method, said second method comprises observing a first maximum temperature of the plurality of temperatures at which the first analyte is detected in the first sample and observing a second maximum temperature of the plurality of temperatures at which the first analyte is detected in the second sample. Selecting the recombinant clone comprises comparing temperatures (either maximum or minimum temperatures) at which fully loaded virions are observed in two or more non-identical viral preparations using the methods of the present disclosure. For example, in some implementations, selecting the recombinant clone comprises comparing the first maximum temperature to the second maximum temperature. In some implementations, the selection of the recombinant clone is based upon the higher of the first maximum temperature and the second maximum temperature. Alternatively, or additionally, selecting the recombinant clone comprises calculating a first ratio of two or more of the first, second, third, or fourth analytes of the first suspension at one temperature of the plurality of temperatures (e.g., the first maximum temperature) and calculating a second ratio of two or more of the first, second, third, or fourth analytes of the second suspension at the one temperature of the plurality of temperatures. A nonlimiting example of a ratio of analytes than can be used to make the selection is a ratio of the quantity of fully filled virions to sum of the quantities of empty virions, partially filled virions, fully filled, and over-filled virions at any given detection temperature.

In another aspect, the present disclosure provides a composition comprising a viral clone selected by any implementation of the second method disclosed herein.

### LIST OF EMBODIMENTS

Embodiment 1 is a method of analyzing recombinant viruses, the method comprising:
   infusing a sample comprising a suspension of recombinant viral particles into a charge detection mass spectrometer, the mass spectrometer comprising an electrospray needle that is maintained at a first temperature for a first period of time;
   while a portion of the sample is disposed in the electrospray needle and after the first period of time, maintaining the electrospray needle at a second temperature for a second period of time;
      wherein the second temperature is higher than the first temperature;
   during the first period of time, detecting a first analyte having a first mass-to-charge (m/z) ratio and having a first charge (z); and
   during the second period of time, detecting the first analyte.
Embodiment 2 is the method of Embodiment 1, further comprising:
   while the portion is disposed in the electrospray needle, and after the first period of time and the second period of time, maintaining the electrospray needle at a third temperature for a third period of time;
      wherein the third temperature is higher than the second temperature; and during the third period of time, detecting the first analyte.
Embodiment 3 is the method of Embodiment 1 or Embodiment 2, wherein detecting the first analyte further comprises quantifying the first analyte.
Embodiment 4 is the method of any one of the preceding Embodiments, further comprising:
   during the first period of time, the second period of time or the third period of time, detecting a second analyte having a second mass-to-charge (m/z) ratio and having a second charge (z).
Embodiment 5 is the method of Embodiment 4, wherein detecting the second analyte further comprises quantifying the second analyte.
Embodiment 6 is the method of any one of the preceding Embodiments, further comprising:
   during the first period of time, the second period of time or the third period of time, detecting a third analyte having a third mass-to-charge (m/z) ratio and having a third charge (z).
Embodiment 7 is the method of Embodiment 6, wherein detecting the third analyte further comprises quantifying the third analyte.
Embodiment 8 is the method of any one of the preceding Embodiments, wherein the first analyte, second analyte, and/or the third analyte each is an ionized analyte selected from a group consisting of a fully filled virion, a partially filled virion, an empty virion, and an over-filled virion.
Embodiment 9 is the method of any one of the preceding Embodiments, wherein the first temperature is greater than or equal to 0° C.
Embodiment 10 is the method of any one of the preceding Embodiments, wherein the second temperature or the third temperature is less than or equal to 100° C.
Embodiment 11 is the method of any one of the preceding Embodiments, further comprising prior to the infusing the sample into the mass spectrometer, contacting the sample with an effective amount of an environmental stress agent to destabilize the viral particles.
Embodiment 12 is the method of any one of Embodiments 1 through 10, further comprising while the portion of the sample is disposed in the electrospray needle, contacting the sample the effective amount of the environmental stress agent.
Embodiment 13 is the method of Embodiment 11 or Embodiment 12, wherein the environmental stress agent is selected from a group consisting of an acid, a base, a salt, an oxidizer reagent, and a combination of any two or more of the foregoing environmental stress agents.
Embodiment 14 is the method of any one of the preceding Embodiments, wherein first period of time is about 30 seconds to about 20 minutes.
Embodiment 15 is the method of any one of the preceding Embodiments, wherein second period of time is about 30 seconds to about 20 minutes.
Embodiment 16 is the method of any one of the preceding Embodiments, wherein third period of time is about 30 seconds to about 20 minutes.
Embodiment 17 is the method of any one of the preceding Embodiments, wherein the viral particles comprise Adeno-associated viral particles.
Embodiment 18 is a method of selecting a recombinant viral clone to for gene therapy use, the method comprising:
   analyzing a first suspension of recombinant viral particles, wherein analyzing the first suspension comprises:
      infusing a first sample comprising a first suspension of recombinant viral particles from a first clone into an electrospray needle of a charge detection mass spectrometer; wherein the electrospray needle is maintained at an initial temperature;
      while the first sample is the electrospray needle, sequentially heating the electrospray needle to a plurality of temperatures, each of the plurality of temperatures being successively higher than the initial temperature;
      wherein the electrospray needle is maintained at the initial temperature and at each successive temperature of the plurality of temperatures for a period of time;
      while the first sample is disposed in the electrospray needle at each of the plurality of temperatures, detecting a first analyte having a first mass-to-charge (m/z) ratio and having a first charge (z); and
      observing a first maximum temperature of the plurality of temperatures at which the first analyte is detected in the first sample;
   analyzing a second suspension of recombinant viral particles, wherein analyzing the second suspension comprises:
      infusing a second sample comprising a second suspension of recombinant viral particles from a second clone into the electrospray needle of the charge detection mass spectrometer; wherein the electrospray needle is maintained at the initial temperature;
      while the second sample is the electrospray needle, sequentially heating the electrospray needle to the plurality of temperatures;
      wherein the electrospray needle is maintained at the initial temperature and each of the successive temperatures for the period of time;
      while the second sample is disposed in the electrospray needle at each of the plurality of temperatures, detecting the first analyte; and
      observing a second maximum temperature of the plurality of temperatures at which the first analyte is detected in the second sample;
   wherein selecting the recombinant clone comprises comparing the first highest temperature to the second maximum temperature.
Embodiment 19 is the method of Embodiment 18, wherein detecting the first analyte further comprises quantifying the first analyte, wherein selecting the recombinant clone comprises comparing a quantity of the first analyte observed in the first sample at the first maximum temperature to a quantity of the first analyte observed in the second sample at the second maximal temperature.
Embodiment 20 is the method of Embodiment 18 or Embodiment 19, further comprising:
   while the first sample is disposed in the electrospray needle at each of the plurality of temperatures, detecting a second analyte having a second mass-to-charge (m/z) ratio and having a second charge (z);
   while the second sample is disposed in the electrospray needle at each of the plurality of temperatures, detecting the second analyte;
   wherein detecting the second analyte comprises quantifying the second analyte; and
   wherein selecting the recombinant clone comprises comparing a quantity of the second analyte observed in the first sample at the first maximum temperature to a quantity of the second analyte observed in the second sample at the second maximal temperature.
Embodiment 21 is the method of any one of Embodiments 18 through 20 further comprising:
   while the first sample is disposed in the electrospray needle at each of the plurality of temperatures, detecting a third analyte having a second mass-to-charge (m/z) ratio and having a second charge (z);
   while the second sample is disposed in the electrospray needle at each of the plurality of temperatures, detecting a third analyte having a third mass-to-charge (m/z) ratio and having a third charge (z);
   wherein selecting the recombinant clone comprises comparing a quantity of the third analyte observed in the first sample at the first maximum temperature to a quantity of the third analyte observed in the second sample at the second maximal temperature.
Embodiment 22 is the method of any one of Embodiments 18 through 21, wherein the first analyte, second analyte, and/or the third analyte each is an ionized analyte selected from a group consisting of a fully filled virion, a partially filled virion, an empty virion, and an over-filled virion.
Embodiment 23 is the method of any one of claims 18 through 22, further comprising:
   calculating a first ratio equal to the quantity of the first analyte observed at the first maximum temperature divided by a sum of the quantities of the second and third analytes observed at the first maximum temperature;
   calculating a second ratio equal to the quantity of the first analyte observed at the second maximum temperature divided by a sum of the quantities of the second and third analytes observed at the second maximum temperature;
   wherein selecting the recombinant clone comprises comparing the first ratio to the second ratio.
Embodiment 24 is the method of any one of the preceding Embodiments, wherein the first temperature, the second temperature, and the third temperature (if used) are within a temperature range of 15° C. to 75° C.
Embodiment 25 is the method of any one of the preceding Embodiments, wherein the first temperature, the second temperature, and the third temperature (if used) are within a temperature range of 15° C. to 65° C.
Embodiment 26 is the method of any one of the preceding Embodiments, wherein the first temperature, the second temperature, and the third temperature (if used) are within a temperature range of 15° C. to 35° C.
Embodiment 27 is the method of any one of the preceding Embodiments, wherein a temperature differential between any two of the plurality of temperatures is 5° C.
Embodiment 28 is the method of any one of the preceding Embodiments, wherein a temperature differential between any two of the plurality of temperatures is 10° C.
Embodiment 29 is a composition comprising a recombinant viral clone selected by the method of any one of Embodiments 18 through 23.

### EXAMPLES

The following examples are provided solely to illustrate the present invention and are not intended to limit the scope of the invention, described herein.

### Example 1. Characterization of AAV Suspensions Using CD-MS Analysis

Materials. CMV-GFP filled AAV2 capsids were purchased from Virovek. Empty AAV8 and mixed empty, partially filled, and fully filled capsids were provided by Regenxbio. Production of the empty AAV8 capsids was as described in Kostelic, M. M.; Zak, C. K.; Liu, Y.; Chen, V. S.; Wu, Z.; Sivinski, J.; Chapman, E.; Marty, M. T., UniDecCD: Deconvolution of Charge Detection-Mass Spectrometry Data. Anal. Chem. 2021, 93 (44), 14722-14729; hereinafter, “Kostelic 1”; which is incorporated herein by reference in its entirety. Ammonium acetate was purchased from Sigma Aldrich (St. Louis, MO), and 100 kDa Molecular weight cutoff (MWCO) filters were purchased from Sartorius (Bohemia, NY).

Borosilicate glass capillaries coated with PEG were used to produce ESI needles using a Sutter Instruments pipette puller (P-1000) that were then manually clipped. Glass capillaries were purchased from World Precision Instruments. Needles (0.1-micron) were used to resolve intact AAV8 capsids with native MS. The methods used for PEG coating and production of 0.1-micron ESI needles are described in for example, Kostelic, M. M. et al, Surface Modified Nano-Electrospray Needles Improve Sensitivity for Native Mass Spectrometry, J. Am. Soc. Mass Spectrom., 33:1031-1037 (2022), which is incorporated herein by reference in its entirety.

Thermal Temperature Ramps. Empty AAV8 capsid thermal ramps were completed by acquiring mass spectra at temperature range of 15-65° C. in 10° C. increments then 65-75° C. in 5° C. increments. Filled AAV2 thermal ramps were completed at a temperature range of 15-35° C. in 10° C. increments then at a temperature range of 35-75° C. in 5° C. increments. The thermal ramp of the mixed AAV8 with empty, partially filled, and fully filled capsids was completed with a temperature range 15-55° C. in 10° C. increments and then at a temperature range of 55-75° C. in 5° C. increments.

Charge Detection-Mass Spectrometry. CD-MS analysis was performed on a Thermo Scientific UHMR Q-Exactive Orbitrap mass spectrometer (Bremen, Germany) using the method described by Wörner et. al. (“Resolving heterogeneous macromolecular assemblies by Orbitrap-based single-particle charge detection mass spectrometry.” Nat. Methods. 2020, 17 (4), 395-398; which is incorporated herein by reference in its entirety) that measures the signal intensity of single ions to measure the charge with the exception that we used the signal-to-noise (S/N) ratio, rather than the raw signal intensity, to get the charge, as described in Kostelic 1. The parameters for CD-MS analysis were the same for the native MS analysis except the resolution was set to 240,000 for empty AAV capsids. The resolution was lowered 120,000 for filled and mixes of different AAV capsid populations to acquire more scans of single ions because the transient time. Also, the max injection time was variable from 100-500 ms to achieve the optimal number of ions per scan. For empty AAV capsids, a sufficient number (e.g., about 5,000) of ions were collected to provide an accurate CD-MS measurement within the range 18,000-35,000 m/z and 100-200 charges. For Filled AAV capsids, approximately 3,000 ions per measurement were collected. Because the many ions can be collected in a single spectrum, a CD-MS measurements were completed in 1-3 minutes at lower temperatures. However, at higher temperatures, acquisitions of 5-10 minutes were performed to collect enough ions for an accurate mass assignment.

CD-MS Data Processing and Deconvolution. CD-MS mass spectra was processed and deconvolved using UniDecCD (UCD) described in Kostelic 1. For calibrating the S/N to charge slope, we overlayed the CD-MS deconvolved mass spectrum with the native deconvolved mass spectrum of empty AAV8 and chose a slope that made the center of the mass distribution the same for both. At 240,000 resolution, a slope of 0.2193 S/N per charge was used, and at 120,000 resolution a slope of 0.1566 S/N per charge was used.

For the CD-MS processing, the m/z bin size was set to 15, the charge bin size was set to 1. Gaussian smoothing in the m/z dimension was set to 7, and in the charge dimension it was set to 5. For the deconvolution, the m/z FWHM was set to 20, and the charge FWHM was set to 15. The point smooth width was set to 5 and the smooth charge sates feature was turned off. These parameters provided well resolved CD-MS deconvolved mass spectra. For, CD-MS intensity vs temperature plots, CD-MS deconvolved mass peaks were extracted based on their local max with a window of 100 kDa.

### Example 2. Characterization of Environmentally Stressed AAV Suspensions Using CD-MS Analysis

AAV suspensions were stressed as described in Kostelic, M. M. et al, Stability and Dissociation of Adeno-Associated Viral Capsids by Variable Temperature-Charge Detection-Mass Spectrometry, Analytical Chemistry 94:11723-11727 (2022), which is incorporated herein by reference in its entirety. Subsequently, the suspensions were subjected to CD-MS characterization and analysis as described for non-stressed preparations in Example 1.

Stressed AAV Preparations. pH stressed capsids devoid of nucleic acid cargo were buffer exchanged into pH 3 or pH 5 ammonium acetate with acetic acid. Stressed capsids were left in pH 3 or pH 5 buffer for at least 30 minutes at 4° C. prior to analysis.

All publications mentioned herein are incorporated by reference to the extent they support the present invention.

### REFERENCES

A number of patents and publications are cited above in order to more fully describe and disclose the invention and the state of the art to which the invention pertains. Each of these references is incorporated herein by reference in its entirety into the present disclosure, to the same extent as if each individual reference was specifically and individually indicated to be incorporated by reference.

1. A method of analyzing recombinant viruses, the method comprising:
infusing a sample comprising a suspension of recombinant viral particles into a charge detection mass spectrometer, the mass spectrometer comprising an electrospray needle that is maintained at a first temperature for a first period of time;
while a portion of the sample is disposed in the electrospray needle and after the first period of time, maintaining the electrospray needle at a second temperature for a second period of time;
wherein the second temperature is higher than the first temperature;
during the first period of time, detecting a first analyte having a first mass-to-charge (m/z) ratio and having a first charge (z); and during the second period of time, detecting the first analyte.

2. The method of claim 1, further comprising:
while the portion is disposed in the electrospray needle, and after the first period of time and the second period of time, maintaining the electrospray needle at a third temperature for a third period of time; wherein the third temperature is higher than the second temperature; and during the third period of time, detecting the first analyte.

3. The method of claim 1, wherein detecting the first analyte further comprises quantifying the first analyte.

4. The method of claim 2, further comprising:
during the first period of time, the second period of time or the third period of time, detecting a second analyte having a second mass-to-charge (m/z) ratio and having a second charge (z).

5. The method of claim 4, wherein detecting the second analyte further comprises quantifying the second analyte.

6. The method of claim 2, further comprising:
during the first period of time, the second period of time or the third period of time, detecting a third analyte having a third mass-to-charge (m/z) ratio and having a third charge (z).

7. The method of claim 6, wherein detecting the third analyte further comprises quantifying the third analyte.

8. The method of claim 6, wherein the first analyte, second analyte, and/or the third analyte each is an ionized analyte selected from a group consisting of a fully filled virion, a partially filled virion, an empty virion, and an over-filled virion.

9. (canceled)

10. (canceled)

11. The method of claim 1, further comprising prior to the infusing the sample into the mass spectrometer, contacting the sample with an effective amount of an environmental stress agent to destabilize the viral particles.

12. The method of claim 1, further comprising while the portion of the sample is disposed in the electrospray needle, contacting the sample the effective amount of the environmental stress agent.

13. The method of claim 11, wherein the environmental stress agent is selected from a group consisting of an acid, a base, a salt, an oxidizer reagent, and a combination of any two or more of the foregoing environmental stress agents.

14. The method of claim 1, wherein the first period of time is about 30 seconds to about 20 minutes; or the second period of time is about 30 seconds to about 20 minutes.

15. (canceled)

16. The method of claim 2, wherein the third period of time is about 30 seconds to about 20 minutes.

17. The method of claim 1, wherein the viral particles comprise Adeno-associated viral particles.

18. A method of selecting a recombinant viral clone, the method comprising:
analyzing a first suspension of recombinant viral particles, wherein analyzing the first suspension comprises:
infusing a first sample comprising a first suspension of recombinant viral particles from a first clone into an electrospray needle of a charge detection mass spectrometer; wherein the electrospray needle is maintained at an initial temperature;
while the first sample is the electrospray needle, sequentially heating the electrospray needle to a plurality of temperatures, each of the plurality of temperatures being successively higher than the initial temperature;
wherein the electrospray needle is maintained at the initial temperature and at each successive temperature of the plurality of temperatures for a period of time;
while the first sample is disposed in the electrospray needle at each of the plurality of temperatures, detecting a first analyte having a first mass-to-charge (m/z) ratio and having a first charge (z); and
observing a first maximum temperature of the plurality of temperatures at which the first analyte is detected in the first sample;
analyzing a second suspension of recombinant viral particles, wherein analyzing the second suspension comprises:
infusing a second sample comprising a second suspension of recombinant viral particles from a second clone into the electrospray needle of the charge detection mass spectrometer; wherein the electrospray needle is maintained at the initial temperature;
while the second sample is the electrospray needle, sequentially heating the electrospray needle to the plurality of temperatures;
wherein the electrospray needle is maintained at the initial temperature and each of the successive temperatures for the period of time;
while the second sample is disposed in the electrospray needle at each of the plurality of temperatures, detecting the first analyte; and
observing a second maximum temperature of the plurality of temperatures at which the first analyte is detected in the second sample;
wherein selecting the recombinant clone comprises comparing the first highest temperature to the second maximum temperature.

19. The method of claim 18, wherein detecting the first analyte further comprises quantifying the first analyte, wherein selecting the recombinant clone comprises comparing a quantity of the first analyte observed in the first sample at the first maximum temperature to a quantity of the first analyte observed in the second sample at the second maximal temperature.

20. The method of claim 18, further comprising: while the first sample is disposed in the electrospray needle at each of the plurality of temperatures, detecting a second analyte having a second mass-to-charge (m/z) ratio and having a second charge (z); while the second sample is disposed in the electrospray needle at each of the plurality of temperatures, detecting the second analyte; wherein detecting the second analyte comprises quantifying the second analyte; and wherein selecting the recombinant clone comprises comparing a quantity of the second analyte observed in the first sample at the first maximum temperature to a quantity of the second analyte observed in the second sample at the second maximal temperature.

21. The method of claim 18, further comprising: while the first sample is disposed in the electrospray needle at each of the plurality of temperatures, detecting a third analyte having a second mass-to-charge (m/z) ratio and having a second charge (z); while the second sample is disposed in the electrospray needle at each of the plurality of temperatures, detecting a third analyte having a third mass-to-charge (m/z) ratio and having a third charge (z); wherein selecting the recombinant clone comprises comparing a quantity of the third analyte observed in the first sample at the first maximum temperature to a quantity of the third analyte observed in the second sample at the second maximal temperature.

22. The method of claim 18, wherein the first analyte, second analyte, and/or the third analyte each is an ionized analyte selected from a group consisting of a fully filled virion, a partially filled virion, an empty virion, and an over-filled virion.

23. The method of claim 18, further comprising:
calculating a first ratio equal to the quantity of the first analyte observed at the first maximum temperature divided by a sum of the quantities of the second and third analytes observed at the first maximum temperature; calculating a second ratio equal to the quantity of the first analyte observed at the second maximum temperature divided by a sum of the quantities of the second and third analytes observed at the second maximum temperature; wherein selecting the recombinant clone comprises comparing the first ratio to the second ratio.

24. (canceled)
